(19) United States
(12) Patent Application Publication
(10) Pub. No.: US 2006/0292666 A1
(43) Pub. Date: Dec. 28, 2006

(54) Fermentation method for the preparation of testolactone by fusarium species

(75) Inventors: Ivan Gale Gilbert, Kalamazoo, MI (US); Michael Jon White, Portage, MI (US)

Correspondence Address: PFIZER INC, 150 EAST 42ND STREET, 5TH FLOOR - STOP 49, NEW YORK, NY, 10017-5612, US

(73) Assignee: PFIZER INC

(21) Appl. No.: 10/573,636

(22) PCT Filed: Sep. 13, 2004
(86) PCT No.: PCT/IB04/02993

§ 371 Date: Mar. 28, 2006

Related U.S. Application Data
(60) Provisional application No. 60/506,945, filed on Sep. 29, 2003.

(51) Int. Cl. C12P 1/02; C12P 33/16

(52) U.S. Cl. 435/55; 435/171; 424/725

(57) ABSTRACT

The present invention relates to a microbial method for the simultaneous dehydrogenation and oxidation of 4-an-drosten-3,17-dione to produce 17a-oxo-D-homo-1,4-andrustadiene-3,17-dione in high yield and high substrate concentrations, by means of filamentous Fusarium species.

### BACKGROUND OF THE INVENTION

1. Field of the Invention

The present invention relates to a microbial method for the simultaneous dehydrogenation and oxidation of 4-androsten-3,17-dione, Formula I, 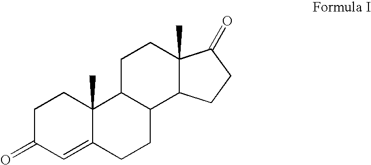
to produce 17α-oxo-D-homo-1,4-androstadiene-3,17-dione, Formula II, in high yield.

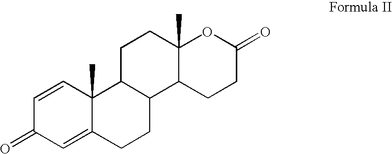

2. Background of the Invention

Testolactone is an antineoplastic used to treat breast cancer.

U.S. Pat. Nos. 2,744,120 and 2,823,171 relates to microbial processes for the conversion of a variety of substrates to testolactone, also referred to as 1-dehydrotestolactone or 17α-oxo-D-homo-1,4-androstadiene-3,17-dione, using Cylindrocarpon and Fusarium species. When the Fusanium species is used with progesterone as the substrate, many products, including testolactone and 1α-hydroxy testolactone, are produced. The examples discuss reactions in which the substrate concentration is 0.5 g/L or less.

G.B. Patent No. 1,220,829 relates to the use of Fusarium species for the microbial preparation of testolactone using 16-dehydropregnenolone and 16-dehydropregnenolone acetate as substrates. The examples in this patent use substrate concentrations of 0.5 g/L or less.

Socic, H. et. al., Z. Anal. Chem. 1968, 243, 291 relates to separation and identification of steroids by fermentative oxidation of progesterone using various Fusarium species. Multiple products were identified.

Despite the above reports, there remains a need for an improved method for the preparation of testolactone.

### DESCRIPTION OF THE INVENTION

Testolactone (17α-oxo-D-homo-1,4-androstadiene-3,17-dione) is an antineoplastic used to treat some cases of breast cancer in females. The disclosed microbial transformation methods described herein are used to convert the low-cost commodity steroid, 4-androsten-3,17-dione (I) to17α-oxo-D-homo-1,4-androstadiene-3,17-dione (II), in yields greater than 85% and at substrate concentrations as high as 80 g/liter.

Any filamentous fungus of the genus Fusarium capable of simultaneous dehydrogenation and oxidation of 4-androsten-3,17-dione (I) to produce 17α-oxo-D-homo-1,4-androstadiene-3,17-dione (II) in high yield can be used in the invention process. The methods described in the examples may be used to determine the suitability of the filamentous fungus of the genus Fusarium. Preferably, Fusarium solani is used. More preferably, Fusariun solani ATCC 46829 is used.

The fungal enzymes may be utilized in the form of an actively growing culture or a whole-cell concentrate. The conversion is carried out in a suitable bioconversion medium. If the conversion is conducted using a whole cell concentrate, the bioconversion medium is similar to a culture medium, with the carbon and nitrogen sources omitted. The steroid to be converted is added in the same manner as it would be added to a submerged culture. Preferably the fungus is grown in submerged culture where the culture medium serves as the bioconversion medium under aerobic conditions using any art-recognized procedure, and the transformation performed in situ. More preferably, the desired fungus is grown in submerged culture under aerobic conditions as set forth below and, more specifically, as set forth in EXAMPLES 1 and 2 using the ingredients specified, or other suitable carbon and nitrogen sources as are known to those skilled in the art. Non-limiting examples of suitable carbon sources include monosacharides, disaccharides, trisacharides and sugar alcohols such as glycerol and glucitol. Non-limiting examples of suitable organic nitrogen sources include casein, corn steep liquor, meat extract, fishmeal and soy protein hydrolysate. Non-limiting examples of suitable inorganic nitrogen sources include potassium nitrate, ammonium chloride, sodium nitrite and the like. Generally a primary and secondary vegetative seed procedure is used in preparation for the fungal transformation of 4-androsten-3,17-dione (I) to 17α-oxo-D-homo-1,4-androstadiene-3,17-dione (II). Alternatively, a primary vegetative seed can be used directly to inoculate bioconversion media.

Primary vegetative seed cultures may be incubated for a period of 24 to 96 hours (preferably 48 hours) at a temperature between 20° and 37° (preferably 28°), and an initial pH between 3.0 and 8.0. Secondary vegetative seed medium is inoculated with 0.006% to 0.1% (v/v) primary vegetative seed culture, but typically 0.012% (v/v), and incubated for a period of 36 to 72 hours (preferably 48-60 hours) at a temperature between 20° and 37° (preferably 28°). The pH of the secondary seed medium can be between 3.0 and 8.0, but preferably between 3.0 and 5.0. The bioconversion medium, which can be the same or similar to the secondary vegetative seed medium, is inoculated with 1% to 10% (v/v) secondary vegetative seed culture (preferably 3% to 5%). Bioconversion fermentation conditions can be the same as those used for cultivation of the secondary vegetative seed culture.

After an initial incubation period of zero to 72 hours (preferably 12 to 24 hours), 4-androsten-3,17-dione (I), preferably micronized, is added to the bioconversion culture. Micronized 4-androsten-3,17-dione (I) can be added as a dry powder or an aqueous slurry, either as a single addition, a series of additions, or a continual feed. The micronized 4-androsten-3,17-dione (I) may be used at concentrations between 1 g/L and 80 g/L, between 10 g/L and 80 g/L, between 20 g/L and 80 g/L, and between 40 g/L and 80 g/L. Other concentration ranges such as between 10 g/L and 20 g/L, between 20 g/L and 40 g/L, and between 40 g/L and 60 g/L may also be used. A preferred concentration range for the micronized 4-androsten-3,17-dione (I) is 50 g/L and 70 g/L. Bioconversion of 4-androsten-3,17-dione (I) to form 17α-oxo-D-homo-1,4-androstadiene-3,17-dione (II), is allowed to proceed for between 1 and 7 days.

The rate, and extent, of conversion of 4-androsten-3,17-dione (I) to 17α-oxo-D-homo-1,4-androstadiene-3,17-dione (II) can be greatly improved by: (i) culturing the selected fungus, and performing the bioconversion, in the presence of a detergent. The detergent may be selected from the group consisting of non-ionic detergents, but preferably the sub-groups consisting of ethoxylated alkylphenols and polyoxyethylenesorbitan esters. More preferably, octylphenoxy polyethoxy ethanol is used; (ii) culturing the selected fungus, and performing the bioconversion, in the presence of a natural oil. Non-limiting examples of natural oils include caster oil, corn oil, cottonseed oil, lard oil, linseed oil, olive oil, peanut oil, rapeseed oil, safflower seed oil, soybean oil, sunflower seed oil, beef tallow, palm oil, cod liver oil, whale oil, shark oil, neats foot oil and wheat germ oil. Preferably, soybean oil is used; (iii) using a combination of the methodologies identified in (i) and (ii).

Once the conversion of 4-androsten-3,17-dione (I) to 17α-oxo-D-homo-1,4-androstadiene-3,17-dione (II) is complete, 17α-oxo-D-homo-1,4-androstadiene-3,17-dione (II) can be isolated using any one of a number of art-recognized procedures. Preferably, filtered or centrifuged beer solids are extracted using an organic solvent, such as methanol, acetone, butyl acetate, or methylene chloride, and the 17α-oxo-D-homo-1,4-androstadiene-3,17-dione (II) is isolated by crystallization. The crystallization solvents include a solvent selected from, but not restricted to, the group consisting of water, methanol, acetone, butyl acetate, methylene chloride, or combinations thereof. The preferred extraction solvent is methylene chloride and preferred crystallization solvent is n-butyl acetate.

### Definitions

The definitions and explanations below are for the terms as used throughout this entire document including both the specification and the claims.

All temperatures are in-degrees Celsius.

r.p.m. refers to revolutions per minute.

TLC refers to thin-layer chromatography.

HPLC refers to high-pressure liquid chromatography.

psig refers to pounds per square inch gage.

DO refers to dissolved oxygen.

RO refers to reverse osmosis.

SLM refers to standard liters per minute.

VVM refers to volume per minute.

OUR refers to oxygen uptake rate.

When solvent mixtures are used, the ratios of solvents used are volume/volume (v/v).

When the solubility of a solid in a solvent is used the ratio of the solid to the solvent is weight/volume (wt/v).

### EXAMPLES

Without further elaboration, it is believed that one skilled in the art can, using the preceding descriptions, practice the present invention to its fullest extent. The following detailed examples describe how to prepare the various compounds and/or perform the various processes of the invention and are to be construed as merely illustrative, and not limitations of the preceding disclosure in any way whatsoever.

Those skilled in the art will promptly recognize appropriate variations from the procedures both as to reactants and as to reaction conditions and techniques.

### Example 1

Bioconversion of 4-androsten-3,17-dione (I) to 17α-oxo-D-homo-1,4-androstadiene-3,17-dione (II) using a submerged culture of Fusarium solani ATCC46829 at a 10-L fermentation scale.

### (A) Primary-Seed Stage

Frozen vegetative cells of Fusarium solani ATCC46829 were thawed, transferred to potato-dextrose-agar plates (PDA), and incubated at 28° for 72 hours. Single mycelia-plugs (6-7 mm diam.) were used to inoculate siliconized 500-mL stippled shake flasks containing 100 mL primary-seed medium. Primary-seed medium consists of (per liter of RO water): dextrin, 50 g; soy flour, 35 g; cerelose, 5 g; coboalt chloride hexahydrate, 2 mg; silicone defoamer (SAG 471), 0.5 mL; pre-sterilization pH 7.0-7.2, adjusted with sodium hydroxide (2N). Fusarium solani ATCC46829 was incubated for 48 hours at 28°, using a controlled-environment incubator-shaker set at 270 rpm. (2″ orbital stroke).

### (B) Secondary-Seed Stage

Ten-liter secondary-seed fermentations were inoculated using 1.2 mL vegetative primary-seed culture (0.012% [v/v] inoculation rate). Secondary-seed medium contains (per liter of RO water): cerelose, 60 g; soyflour, 25 g; soybean oil, 5 mL; magnesium heptahydrate, 1 g; potassium dihydrogen phosphate, 0.74 g; octylphenoxy polyethoxy ethanol, 0.25 mL; silicone defoamer (SAG 471), 0.5 mL; pre-sterilization pH 3.95-4.00, adjusted with concentrated sulfuric acid. The fermentors, containing secondary-seed medium, were sterilized for 20 minutes at 121° using both jacket and injection steam. The agitation rate during sterilization was 200 r.p.m. Post-sterilization, the medium pH was adjusted to 4.0 using sterile sulfuric acid (5%). Fusarium solani ATCC46829 was incubated at 28° using the following initial parameters: agitation, 100 r.p.m.; backpressure=5 psig; airflow=2.5 SLM (0.25 VVM); low DO set point, 50%; pH control, none. When the DO first drops to 50%, the airflow was increased to 5 SLM (0.5 VVM). When the culture reaches low DO again, 50% DO was maintained using agitation control. Secondary-seed cultures were harvested at approximately 52 hours post-inoculation, when the OUR was between 15 and 25 mM/L/h.

### (C) Steroid Bioconversion

Ten-liter steroid-bioconversion fermentations were inoculated using 300 mL vegetative secondary-seed culture (3% [v/v] inoculation rate). Steroid-bioconversion medium was essentially the same as secondary-seed medium, with the exception that octylphenoxy polyethoxy ethanol was increased from 0.25 mL/L to 2.0 mL/L. Sterilization conditions and pH adjustment were as described for secondary-seed medium. Fusarium solani ATCC46829 was incubated at 28° using essentially the same initial parameters as those used for secondary-seed cultivation, with the exception that the initial agitation was 200 r.p.m. At 15 hours post-inoculation, 200 g micronized 4-androsten-3,17-dione (I) slurried in a minimal volume of 0.2% octylphenoxy polyethoxy ethanol was added to the 10-L fermentation.

Bioconversion cultures were assayed on a daily basis for 17α-oxo-D-homo-1,4-androstadiene-3,17-dione (II) using HPLC. One milliliter of whole beer was extracted with 10 mL warm acetonitrile. Cells were separated from the aqueous-acetonitrile mixture by centrifugation (3,000×g for 10 minutes), and 5 μL extract injected onto an HPLC column. Conditions for HPLC were as follows: Spectra-Physics chromatograph fitted with a C18 reverse-phase column (150×4.6 mm) column; column temperature, 30°; mobile phase, acetonitrile/0.25% phosphoric acid (45/55, v/v); flow rate=1 mL/minute; detection, 240 nm; run time=12 minutes.

Bioconversion of 4-androsten-3,17-dione (I) to 17α-oxo-D-homo-1,4-androstadiene-3,17-dione (ED was complete in approximately 2 days.

### (D) Isolation Procedure

The whole beer at harvest, from a 10-L fermentation, was centrifuged and the rich solids were recovered by centrifugation. The rich solids were extracted with 10 liters of methylene chloride. The rich organic extract was separated from the solids by settling. The methylene chloride extract was filtered through diatomaceous earth and concentrated to 500 mL by distillation. 500 mL of n-butyl acetate was added. This mixture was concentrated to 500 mL and cooled to 4° C. to complete product crystallization. The crystals were recovered by filtration, washed with cold butyl acetate to remove color, and dried to give 165 g of crystalline 17α-oxo-D-homo-1,4-androstadiene-3,17-dione (II). Removal of impurities, if necessary, was accomplished by dissolution in methylene chloride and replacing with n-butyl acetate to re-crystallize the product (see EXAMPLE 2).

### Example 2

Bioconversion of 4-androsten-3,17-dione (I) to 17α-oxo-D-homo-1,4-androstadiene-3,17-dione (II) was performed using a submerged culture of Fusarium solani ATCC46829 at a 100-mL fermentation scale.

### (A) Primary-Seed Stage

Primary-seed cultures were prepared as described in EXAMPLE 1.

### (B) Secondary-Seed Stage

One hundred milliliter secondary-seed medium, in a siliconized 500 mL stippled shake flask, was inoculated using a single drop of vegetative primary-seed culture. Secondary-seed medium contains (per liter of RO water): cerelose, 60 g; soy flour, 25 g; soybean oil, 5 mL; magnesium heptahydrate, 1 g; potassium dihydrogen phosphate, 0.74 g; octylphenoxy polyethoxy ethanol, 0.25 mL; silicone defoamer (SAG 471), 0.5 mL; pre-sterilization pH 3.95-4.00, adjusted with concentrated sulfuric acid. Shake flasks, containing secondary-seed medium, were sterilized for 30 minutes at 121° using an autoclave. Fusarium solani ATCC46829 was incubated for 48 hours at 28°, using a controlled-environment incubator-shaker set at 270 rpm. (2″ orbital stroke).

### (C) Steroid Bioconversion

One hundred milliliter steroid-bioconversion medium, in a siliconized 500 mL stippled shake flask, was inoculated using 3 mL vegetative secondary-seed culture (3.0% [v/v] inoculation rate). Steroid-bioconversion medium was essentially the same as the secondary-seed medium, with the exception that octylphenoxy polyethoxy ethanol was increased from 0.25 mL/L to 2.5 mL/L. At 17 hours post-inoculation, 6 g micronized 4-androsten-3,17-dione (I) slurried in a minimal volume of 0.2% octylphenoxy polyethoxy ethanol was added to the 100-mL fermentation. At 24 hours and 48 hours post-inoculation, 2.5 g additional cerelose per 100-mL culture was added.

Bioconversion cultures were assayed on a daily basis for 17α-oxo-D-homo-1,4-androstadiene-3,17-dione (II) using HPLC as described in EXAMPLE 1, with the exception that one-milliliter of whole beer was extracted with 30 mL warm acetonitrile. Bioconversion of 4-androsten-3,17-dione (I) to 17α-oxo-D-homo-1,4-androstadiene-3,17-dione (II) was complete in approximately 7 days.

### (D) Isolation Procedure

The whole beer at harvest, from two 100-mL fermentations, was centrifuged and the rich solids were recovered by centrifugation. The solids were extracted using one liter of methylene chloride. After settling, the beer solids were re-extracted with another liter of methylene chloride. The solids were discarded and the methylene chloride extracts were washed with water, pooled, polished, concentrated by distillation to 50 mL. After adding 100 mL of n-butyl acetate, the mixture was concentrated by distillation to 50 mL and cooled to 4° C. The crystals obtained were recovered by filtration, washed with n-butyl acetate to remove color, and dried to give 10.9 g of crystalline 17α-oxo-D-homo-1,4-androstadiene-3,17-dione (II).

As in EXAMPLE 1, impurity removal (to achieve >99% purity) may be accomplished by re-crystallization. The 10.9 g were dissolved in 60 mL of methylene chloride and then 100 mL of n-butyl acetate was added. This mixture was concentrated to 50 mL and cooled to 4° C. The crystals obtained were recovered by filtration, washed with 15 mL of n-butyl acetate, and dried to give 10.4 g of purified crystalline 17α-oxo-D-homo-1,4-androstadiene-3,17-dione (II).

1. A method for the transformation of 4-androsten-3,17-dione, Formula I, 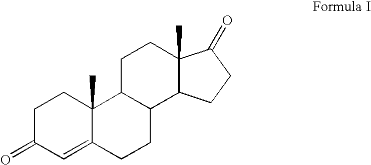
to 17α-oxo-D-homo-1,4-androstadiene-3,17-dione, Formula II 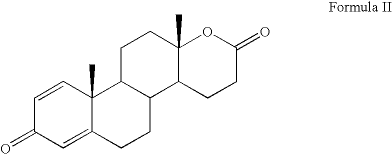
comprising contacting a compound of Formula I in a bioconversion medium with a filamentous species of Fusarium capable of performing the transformation.

2. A method of producing 17α-oxo-D-homo-1,4-androstadiene-3,17-dione according to claim 1 wherein the Fusarium species is Fusarium solani.

3. A method of producing 17α-oxo-D-homo-1,4-androstadiene-3,17-dione according to claim 1 wherein the Fusarium species is Fusarium solani strain ATCC 46829.

4. A method of producing 17α-oxo-D-homo-1,4androstadiene-3,17-dione according to claim 3 wherein the substrate concentration is between 1 g/L and 80 g/L.

5. A method of producing 17α-oxo-D-homo-1,4-androstadiene-3,17-dione according to claim 3 wherein the substrate concentration is between 10 g/L and 80 g/L.

6. A method of producing 17α-oxo-D-homo-1,4-androstadiene-3,17-dione according to claim 3 wherein the substrate concentration is between 20 g/L and 80 g/L.

7. A method of producing 17α-oxo-D-homo-1,4-androstadiene-3,17-dione according to claim 3 wherein the substrate concentration is between 40 g/L and 80 g/L.

8. A method of producing 17α-oxo-D-homo-1,4-androstadiene-3,17-dione according to claim 3 wherein the substrate concentration is between 50 g/L and 70 g/L.

9. A method of producing 17α-oxo-D-homo-1,4-androstadiene-3,17-dione according to claim 3 further comprising the steps of:
a:) preparing a primary seed culture of Fusarium solani ATCC46829;
b) preparing a secondary seed culture from the culture of step a);
c) inoculating a bioconversion medium with the culture of step b);
d) adding micronized 4-androsten-3,17-dione to the bioconversion medium;
e) monitoring the biotransformation for completion;
f) collecting the solids of the bioconversion medium;
g) extracting the solids; and
h) isolating 17α-oxo-D-homo-1,4-androstadiene-3,17-dione.

10. A method according to claims 1-9 wherein the bioconversion medium contains a detergent and a natural oil.

11. A method according to claim 10 wherein the detergent is octylphenoxy polyethoxy ethanol and the natural oil is soybean oil.
